(19) United States
(12) Patent Application Publication
(10) Pub. No.: US 2026/0315259 A1
(43) Pub. Date: Oct. 15, 2026

(54) POWERED EXOSKELETON

(71) Applicants: Mayo Foundation for Medical Education and Research, Rochester, MN (US); Arizona Board of Regents on behalf of Arizona State University, Scottsdale, AZ (US)

(72) Inventors: Kenton R. Kaufman, Rochester, MN (US); Alexander Y. Shin, Rochester, MN (US); Paul H. Kane, Rochester, MN (US); Emily J. Miller, Bangor, WI (US); Griffin A. Mess, Highland, MD (US); Abby Aymond, Scottsdale, AZ (US); Peder C. Solberg, Scottsdale, AZ (US); Thomas Sugar, Tempe, AZ (US); Kevin Hollander, Tucson, AZ (US)

(21) Appl. No.: 19/478,302

(22) PCT Filed: Feb. 28, 2024
(86) PCT No.: PCT/US2024/017674

§ 371 Date: Oct. 24, 2025

Related U.S. Application Data
(60) Provisional application No. 63/462,906, filed on Apr. 28, 2023.

(51) Int. Cl. A61F 5/01

(52) U.S. Cl. CPC A61F 5/013; A61F 2005/0155

(57) ABSTRACT

An elbow joint of a powered orthosis includes a battery, an upper arm attachment configured to be worn on an upper arm of a user, a forearm attachment configured to be worn on a forearm of the user, a drivetrain configured to rotate the forearm attachment relative to the upper arm attachment, and a torque mechanism including a pulley fixed to the forearm attachment, an extension limiter configured to allow for elbow flexion, the extension limiter including a sled that free to move in a first direction and is prevented from moving in a second direction, a sensor configured to receive muscle signals of the user, and a controller configured to receive input from the user and to control rotation of the forearm attachment relative to the upper arm attachment based on the muscle signals.

### CROSS-REFERENCE TO RELATED APPLICATIONS

This application claims the benefit of U.S. Provisional Application Ser. No. 63/462,906 filed Apr. 28, 2023. The disclosure of the prior application is considered part of (and is incorporated by reference in) the disclosure of this application.

### FEDERAL FUNDING STATEMENT

This invention was made with government support under W81XWH-15-2-0071 awarded by the U.S. Army. The government has certain rights in the invention.

### TECHNICAL FIELD

This disclosure relates to powered orthotics that assist individuals with neuromuscular injuries.

### BACKGROUND

Upper limb injuries are frequently seen in sports and motor vehicle accidents. Injuries such as peripheral nerve injury, spinal cord injury, and brachial plexus injury are often debilitating, causing extensive disabilities in the upper extremity. 5% of all motor vehicle accidents result in a form of peripheral nerve injury. About 8% of peripheral nerve injury patients have a brachial plexus injury that results in severe impairment following penetrating wounds, falls, and motor vehicle accidents or other high-energy trauma. Young male adults comprise a majority among patients with a brachial plexus injury. Motor vehicle accidents and falls are a leading cause of spinal cord injuries. 38.1% of all spinal cord injuries between 2010 and 2014 were caused by motor vehicle accidents and 53% were caused by falls. The United States has an estimated annual spinal cord injury incidence of 17,000. The National Spinal Cord Injury Statistical Center estimated 282,000 people were living with a spinal cord injury in 2016. Recovery from the above-mentioned injuries/conditions can be an arduous process involving extensive surgeries and physical rehabilitation. Restoring function in the affected limb is the goal of these rehabilitative approaches. Individuals affected by these injuries are often prescribed some form of orthotic device to support/enable movement in the affected limb during rehabilitation.

### SUMMARY

The systems and methods described herein provide assistance to patients with impaired neuromusculoskeletal function. For example, a user can don an elbow orthosis on an affected side. The elbow orthosis can detect electromyographic (“EMG”) signals and control movement of the user's elbow. Because the EMG signals directly reflect the user's motion intention, the user can neurologically control the orthosis. The elbow orthosis can be lightweight, which can make the orthosis more comfortable for the user. Some components of the orthosis can be placed in a backpack to offload the weight of the system from the affected arm. Additionally, the elbow orthosis can lock in position and support the arm without the need of a constant muscular activation signal. This reduces fatigue and can allow a user to maintain the arm in a locked position.

In an aspect, an elbow joint of a powered orthosis includes a battery, an upper arm attachment configured to be worn on an upper arm of a user, a forearm attachment configured to be worn on a forearm of the user, a drivetrain configured to rotate the forearm attachment relative to the upper arm attachment, the drivetrain including a motor, a belt drive, a brake mechanism connected to the upper arm attachment and the forearm attachment, and a torque mechanism including a pulley fixed to the forearm attachment, a cable routed through the pulley, wherein the cable is fixed to the forearm attachment and the motor such that the cable provides constant torque to the forearm attachment, an extension limiter configured to allow for elbow flexion, the extension limiter including a screw, a nut positioned on the screw, wherein the position of the nut on the screw is variable based on user input, and a sled attached to the cable and configured to move along the screw, wherein the sled is free to move in a first direction along the screw and is prevented from moving in a second direction along the screw by the nut, a surface electromyography sensor configured to receive muscle signals of the user, and a controller configured to receive input from the user and to control rotation of the forearm attachment relative to the upper arm attachment based on the muscle signals received by the surface electromyography sensor.

Embodiments can include one or any combination of two or more of the following features.

In some embodiments, the screw is a ball screw and the nut is a ball screw nut.

In some embodiments, the cable is a bowden cable.

In some embodiments, the battery, the motor, and the belt drive are contained within a housing.

In some embodiments, the housing is configured to be worn on a back of the user.

In some embodiments, the elbow join includes buttons, wherein the buttons are configured to receive input from the user to control the elbow joint.

In some embodiments, a first button allows the user to disengage the solenoid pin from the rotating cam.

In some embodiments, a second button allows the user to change the position of the nut.

In some embodiments, the sled allows for freedom of flexion movement.

In some embodiments, the sled allows user movement to exceed motor speed.

In some embodiments, the motor is located remotely and connected to the forearm attachment by the cable.

In some embodiments, the elbow joint is configured to generate a constant moment around the elbow joint.

In some embodiments, the elbow joint is configured to be worn on a left arm or a right arm.

In some embodiments, the forearm attachment is configured to extend due to gravity.

In some embodiments, the forearm attachment is configured to maintain a position when the motor is powered off.

In some embodiments, the elbow joint includes additional extension limiters to protect from hyperextension.

In some embodiments, the brake mechanism includes a rotating cam rotatably connecting the forearm attachment to the upper arm attachment, and a solenoid pin configured to engage the rotating cam to prevent rotation of the rotating cam in one direction, wherein the rotating cam is free to rotate in the one direction when the solenoid pin disengages the rotating cam.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIGS. 1A-D are a series of illustrations and images that illustrate an elbow orthosis worn by a user.

FIG. 2 is a perspective view of an elbow orthosis.

FIG. 3 is a perspective view of an elbow orthosis.

FIG. 4 is a schematic view of an elbow joint of an elbow orthosis.

FIG. 5 is a schematic view of a drive assembly of an elbow orthosis.

FIG. 6 is a schematic view of a brake assembly of an elbow orthosis.

FIG. 7 is a schematic view of an elbow joint of an elbow orthosis.

FIG. 8 is a schematic view of a drive assembly of an elbow orthosis.

FIG. 9 is a schematic view of an extension limiter of an elbow orthosis.

FIGS. 10A-B are plots of EMG data and resulting logic.

FIGS. 11A-D are plots of varying force levels and received EMG data.

FIG. 12 is a flowchart illustrating a method of controlling an elbow orthosis.

Like reference numbers and designations in the various drawings indicate like elements.

### DETAILED DESCRIPTION

The systems and methods described herein provide assistance to patients with impaired neuromusculoskeletal function. For example, a user can don an elbow orthosis on an affected side. The elbow orthosis can detect electromyographic (“EMG”) signals and control movement of the user's elbow. Because the EMG signals directly reflect the user's motion intention, the user can neurologically control the orthosis.

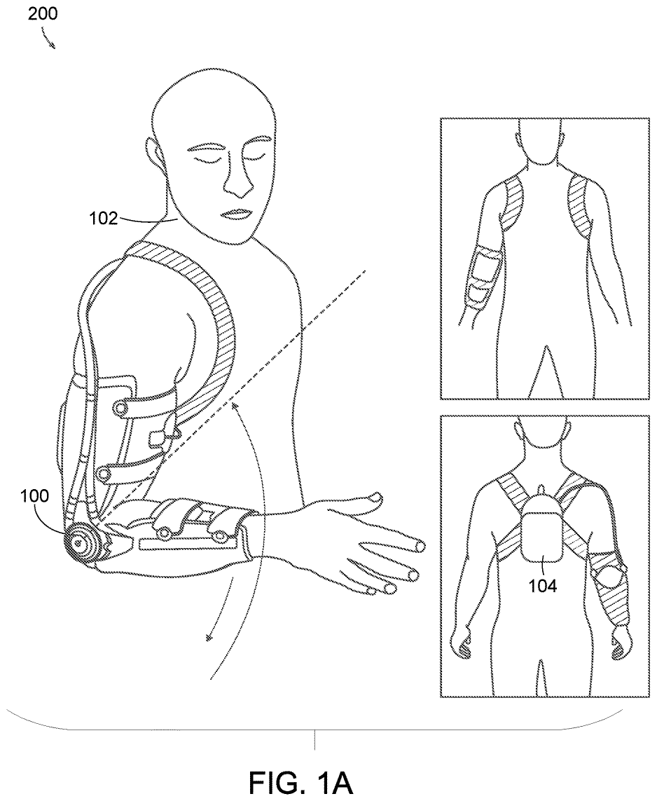

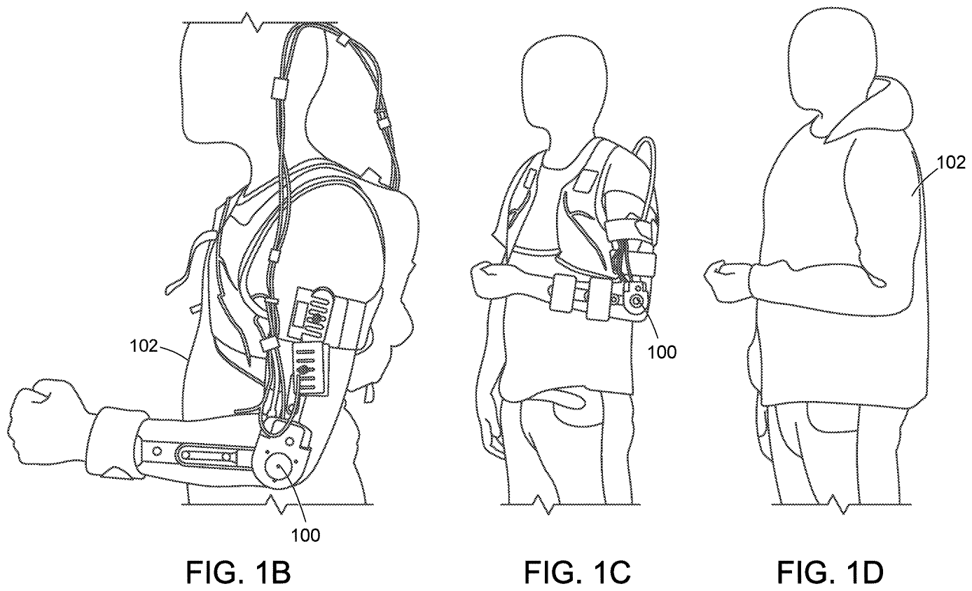

FIGS. 1A-D illustrate an elbow orthosis 100 being worn by a user 102. The elbow orthosis 100 can assist the user 102 with moving his/her elbow. A backpack 104 can contain some components of the elbow orthosis 100 to offload the weight of the components from the arm of the user 102. For example, the backpack 104 can contain a power storage, a drive assembly, etc. Placing the power storage in the backpack 104 can also allow for a larger power storage and a longer battery life for the elbow orthosis.

In some cases, the user 102 may want to cover the elbow orthosis 100. The elbow orthosis 100 can be concealable to allow a user to easily cover the orthosis 100. For example, FIG. 1D illustrates the user 102 covering the elbow orthosis 100 and the backpack 104 by wearing a sweatshirt.

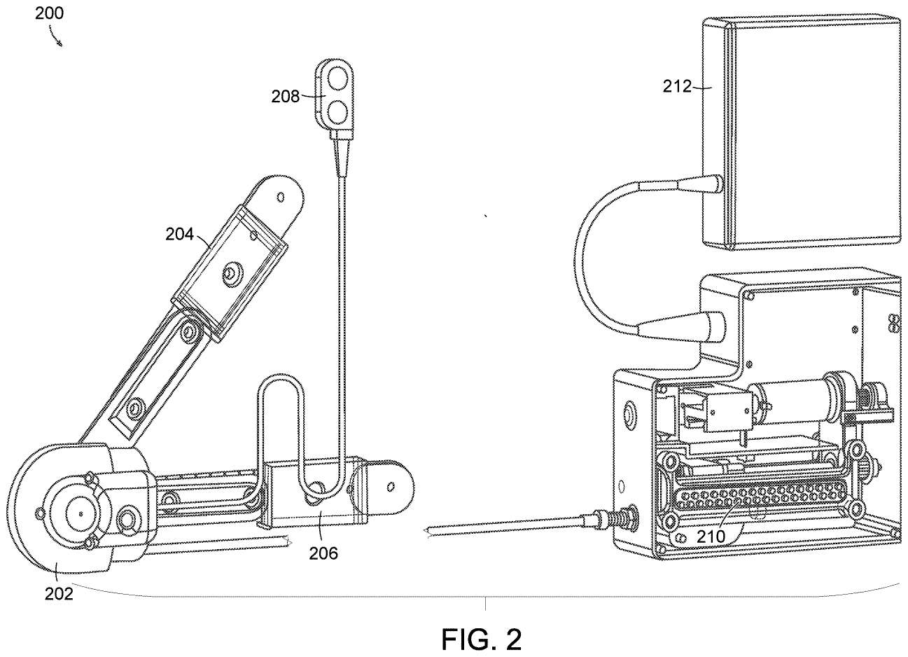

FIG. 2 illustrates an elbow orthosis 200 comprising an elbow joint 202 that connects a forearm component 204 and an upper arm component 206. The orthosis 200 can be worn by a user to assist the user with moving his/her elbow, similar to the orthosis 100 of FIGS. 1A-D. The forearm component 204 can rotate relative to the upper arm component 206 about the elbow joint 202. The upper arm component 206 includes one or more surface EMG sensors 208 that can detect EMG signals from a user. The detected EMG signals can control rotation of the forearm component 204 about the elbow joint 202. For example, a controller can receive the detected EMG signals and control a drive assembly 210 of the elbow orthosis 200. The controller controls the drive assembly 210 to rotate the forearm component 204. A power storage 212 (e.g., a battery) is electrically connected to the elbow orthosis 200 to provide power to the electrical components (e.g., the controller, the drive assembly, the sensors, etc.) of the elbow orthosis 200.

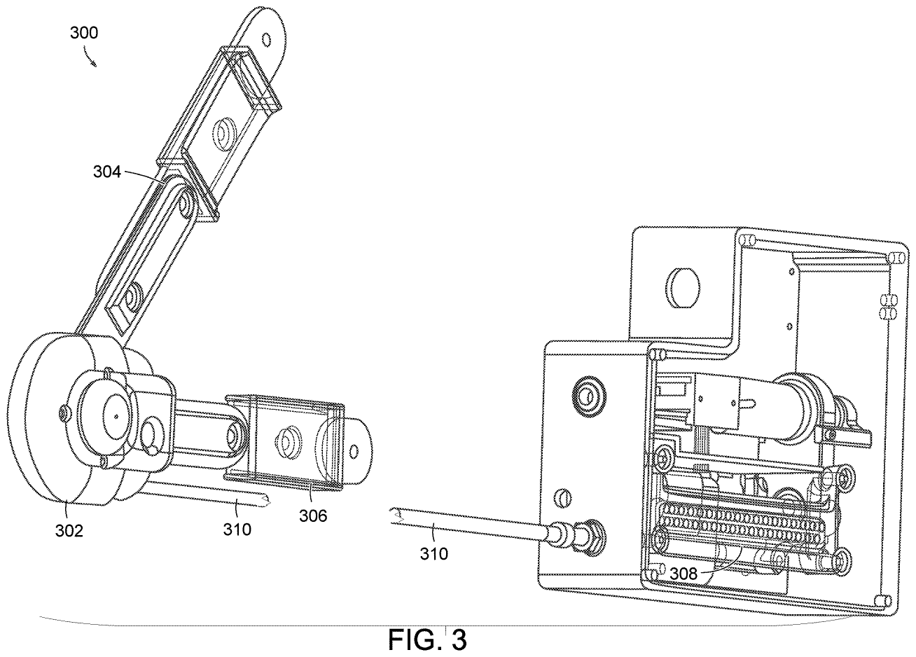

FIG. 3 illustrates an elbow orthosis 300 that is similar to the elbow orthosis 200 of FIG. 2. The power storage and EMG sensors are not illustrated in FIG. 3, but can be implemented with the elbow orthosis 300. An elbow joint 302 connects a forearm component 304 to an upper arm component 306. The forearm component 304 can rotate relative to the upper arm component 306 about the elbow joint 302. A drive assembly 308 is connected to the forearm component 304 by a cable 310. For example, in some embodiments the cable 310 can be a Bowden cable. Although the cable 310 is illustrated as separate pieces, the cable 310 is implemented as a connected cable 310. The cable 310 is looped through the elbow joint 302 such that tension on the cable 310 rotates the forearm component 304 about the elbow joint 302. For example, tension on the cable 310 causes the forearm component 304 to rotate and bends the elbow joint 302, e.g., to form an angle and/or change the angle between the forearm component 304 and the upper arm component 306. This allows a user to flex, i.e., lift, his/her forearm while wearing the orthosis 300. Releasing the tension on the cable 310 allows the elbow joint 302 to straighten so that the forearm component 304 is parallel or nearly parallel to the upper arm component 306. Depending on the position of the user, gravity can help the elbow joint 302 to straighten. This allows a user to extend his/her forearm while wearing the orthosis 300.

The drive assembly 308 can be controlled, e.g., by a controller, to provide tension on the cable 310 and to release tension on the cable 310. Providing and releasing tension on the cable 310 allows the user to flex and extend his/her forearm, as described above. In some implementations, the controller can receive EMG signals, e.g., from EMG sensors, to control the drive assembly 308. This allows a user to neurologically control flexion and extension of his/her forearm while wearing the elbow orthosis.

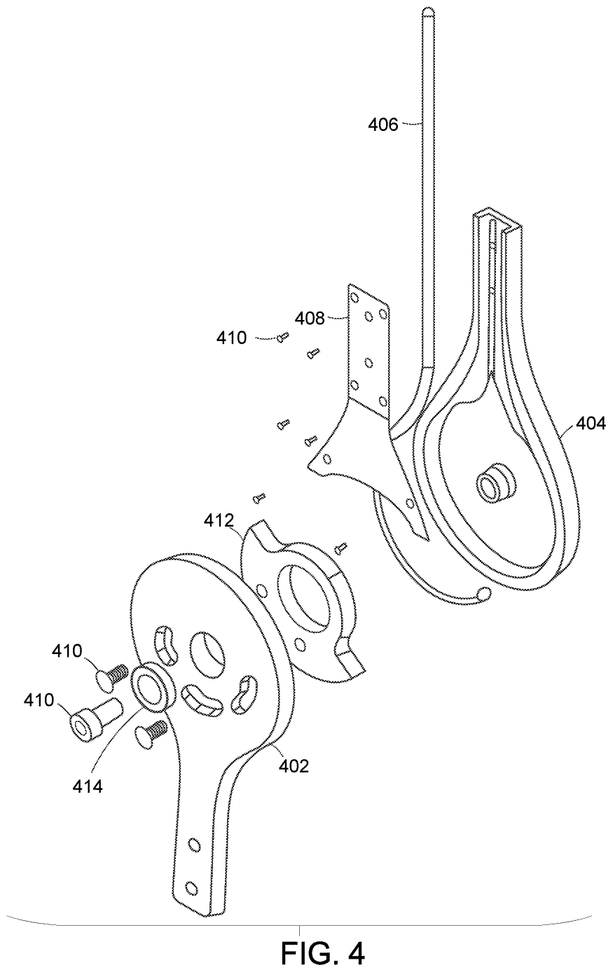

The elbow joint of the orthosis can have a variety of designs to reduce friction between the forearm component and the upper arm component. For example, FIG. 4 illustrates an elbow joint 400 connecting a forearm component 402 and an upper arm component 404. For example, the elbow joint 100 of FIG. 1 can include an elbow joint similar to elbow joint 400. A cover plate 408 is mounted to the upper arm component 404 by fasteners 410 (e.g., screws). A cable 406 (e.g., a Bowden cable) runs between the cover plate 408 and the upper arm component 404. The cable 406 can control the elbow joint to bend and straighten the elbow joint as discussed above. A cam 412 (e.g., a rope cam) and a bearing 414 reduce friction between the forearm component 402 and the upper arm component 404 so that rotation of the forearm component 402 relative to the upper arm component 404 is smooth. Additional fasteners 410 can be used to mount the cam 412, the bearing 414, and the forearm component 402 to the upper arm component 404. Different sizes and types of fasteners 410 can be used to mount different components to each other. The cam can be used to control the torque at the elbow joint. In one instantiation, the cam is used to apply a constant torque about the elbow joint.

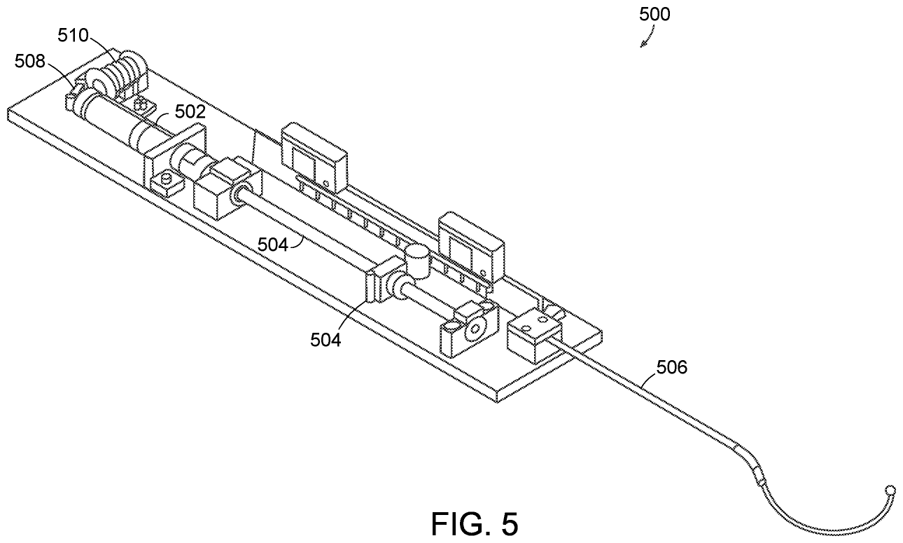

The orthosis includes a drive assembly to control flexion of the elbow. For example, FIG. 5 illustrates a drive assembly 500 that can be used with an elbow orthosis, e.g., the elbow orthosis of FIG. 1A-D or others described herein. The drive assembly 500 can be implemented as a ball-screw drive assembly. The drive assembly includes a motor 502 that is powered by a power source. In some implementations, the motor 502 is a 27 mNm motor with a 16:1 reducer. The motor 502 drives a lead or ball screw 504 to pull a cable 506 (e.g., a Bowden cable). The ball screw 504 can be implemented as a 2 mm pitch. Pulling the cable 506 can provide tension to flex an elbow orthosis (e.g., the elbow orthosis 100 of FIG. 1). The drive assembly also includes a brake detent 508 that can lock the cable 506 (and therefore the elbow orthosis) in position and support the arm without the need of a constant muscular activation signal. This can reduce fatigue and can allow a user to rest the arm in the locked position against gravity. The brake detent 508 can be released by a solenoid 510. A controller can control the solenoid 510 and the brake detent 508 based on user input. For example, buttons connected to the elbow orthosis can receive user input to activate the solenoid 510 or release the solenoid 510, e.g., to activate or release the brake detent 508. The user can control whether the elbow orthosis is locked in position by controlling the brake detent 508.

In some implementations in which the drive assembly is implemented as a linear drive, the linear drive can include a flange that is fixed to a point on the linear ball screw. When the flange arrives at either end of the linear drive, the flange activates a limit switch that automatically brakes the motor 502. In some implementations, the motor can also be attached to an encoder that measures the angular position of the motor 502.

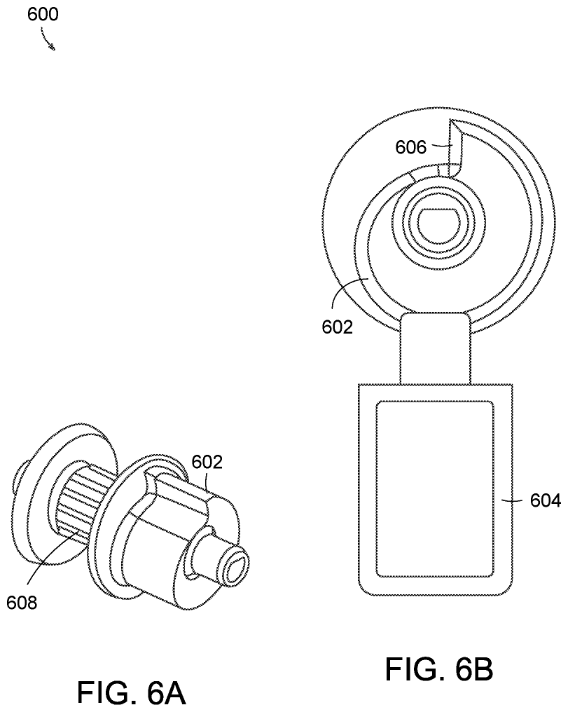

FIG. 6 illustrates an exemplary brake mechanism 600 that can lock a cable, e.g., a cable controlling an elbow orthosis. For example, the brake mechanism 600 can be similar to the brake detent 508 implemented in FIG. 5. The brake mechanism can hold the elbow orthosis at a desired angle without the user needing to constantly provide a muscle activation signal. The brake mechanism 600 includes a cam 602 and a solenoid pin 604.

When the solenoid pin 604 is engaged with the cam 602, the cam 602 can rotate in one direction (associated with elbow flexion) but cannot rotate in the opposite direction (associated with elbow extension). An engaging face 606 of the cam 602 engages the solenoid pin 604 to prevent rotation of the cam 602. The solenoid is a pull solenoid, e.g., the solenoid pin 604 retracts when the solenoid is actuated. Actuation of the solenoid is controlled by a controller, which can receive user input to actuate the solenoid. The system can lock the brake without any input from the user. This can reduce energy expenditure by the user to use the elbow orthosis. When the user wants to extend his/her elbow, user input can retract the solenoid pin 604 (disengaging the brake), allowing free rotation of the brake mechanism and free rotation of the elbow orthosis. A belt drive pulley 608 can connect to a belt drive to drive the cam 602.

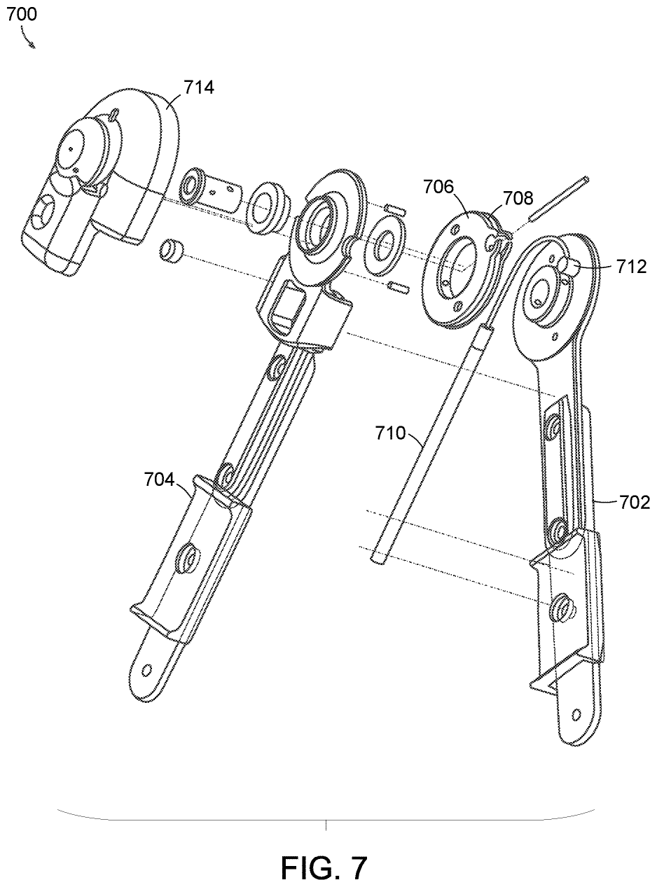

FIG. 7 illustrates an elbow joint 700 of an elbow orthosis including a forearm component 702 and an upper arm component 704. For example, the elbow joint 100 of FIG. 1 can include an elbow joint similar to elbow joint 700. A pulley 706 includes a channel 708 through which a cable 710 (e.g., a Bowden cable) is routed. The cable 710 is attached to a barrel pin seat 712 of the forearm component 702 and can be tensioned to control the elbow joint to bend and straighten the elbow joint as discussed above. A cover 714 can protect the components of the elbow, e.g., from the environment and from wear and tear.

Using a pulley to route the cable 710 to the forearm attachment can provide constant torque to the elbow joint 700 irrespective of an angle of the elbow joint. For example, a constant torque can allow the user to hold a weight in his/her hand, and the torque about the elbow joint will remain at equilibrium. As a non-limiting example, a free body diagram can assume a force of F_{b} is applied by the cable around a radius of the pulley r_{E} when the elbow is flexed at 900 (i.e., the point of highest required torque). A weight Wₒ of about 39 N (approximately the weight of a gallon of milk) can be assumed to be held in a user's hand. The user has a forearm that has a length l_{F} and a weight W_{f}. The moment (F_{b}·r_{E}) required to satisfy the conditions of equilibrium is defined by the equation:

F_{b}·r_{E}=(Wf/2+Wₒ)l_{F}

The radius of the pulley and the constant force applied by the cable can be designed to provide a desired constant torque. For example, using the equation above, the system needs to generate a moment of 10 Nm to 15 Nm at the elbow joint to hold a gallon of milk. The elbow joint 700 can also include cams, bearings, fasteners, etc. as desired to attach the components of the elbow joint 700.

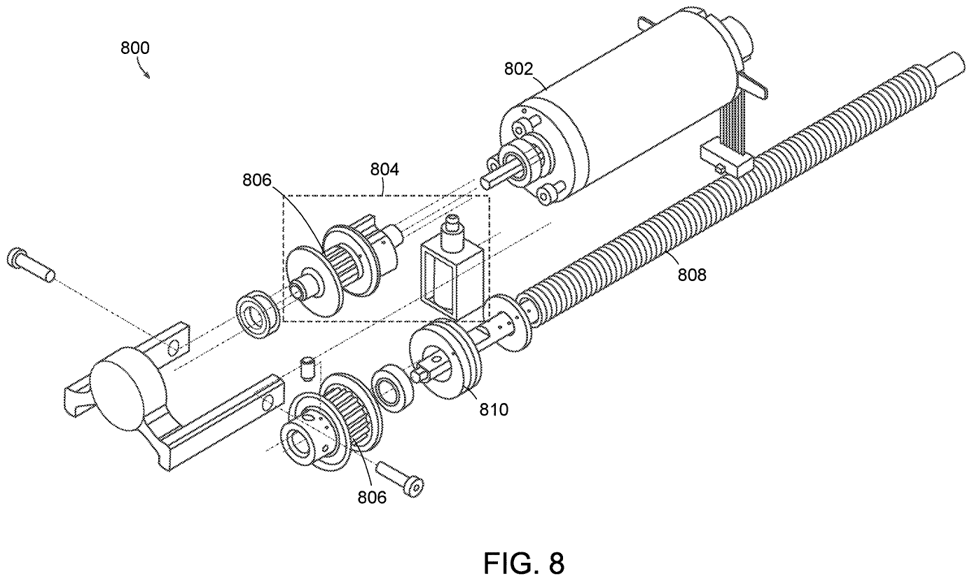

FIG. 8 illustrates an exemplary drivetrain 800 that can provide force in a cable (e.g., the cable 710 of FIG. 7) to provide a constant torque in an elbow joint, as discussed above. The drivetrain 800 includes a motor 802, a brake mechanism 804, a belt drive 806, and a ball-screw 808. The ball-screw 808 converts rotary motion of the motor 802 into linear motion. The brake mechanism 804 attaches to the motor shaft, e.g., with two belt pulleys. The belt drive 806 can attach the motor to the ball-screw while reducing the overall length of the drivetrain. Ball bearings 810 can also be used to provide smooth rotation of the components.

The drivetrain 800 can transfer the linear force created by the ball-screw 808 to a cable (e.g., a cable similar to the cable 710 of FIG. 7) to provide a constant force to the cable and a constant torque to the elbow joint. The extension of the elbow is achieved using the weight of the forearm and is controlled by reverse motion of the motor 802. Mathematically, the linear force in the cable during flexion can be expressed as:

$${F}_{b} = \frac{( 2 π \cdot {M}_{t} \cdot {k}_{b} \cdot e )}{{L}_{s} \cdot 100}$$

Where, Mₜ is the geared motor torque, k_{b} is the belt drive reduction ratio, e is the ball screw's percentage efficiency, and Lₛ is the lead of the ball screw. Using a moment of 15 Nm at the elbow joint, as discussed above:

$$\frac{( 2 π \cdot {M}_{t} \cdot {k}_{b} \cdot e )}{{L}_{s} \cdot 100} \cdot {r}_{E} \geq 15 Nm$$

In some implementations, the elbow joint can be designed to provide at least 40% of the required torque at 120° per second. The motor 802, belt drive 806, and ball screw 808 can be selected to satisfy the equations above.

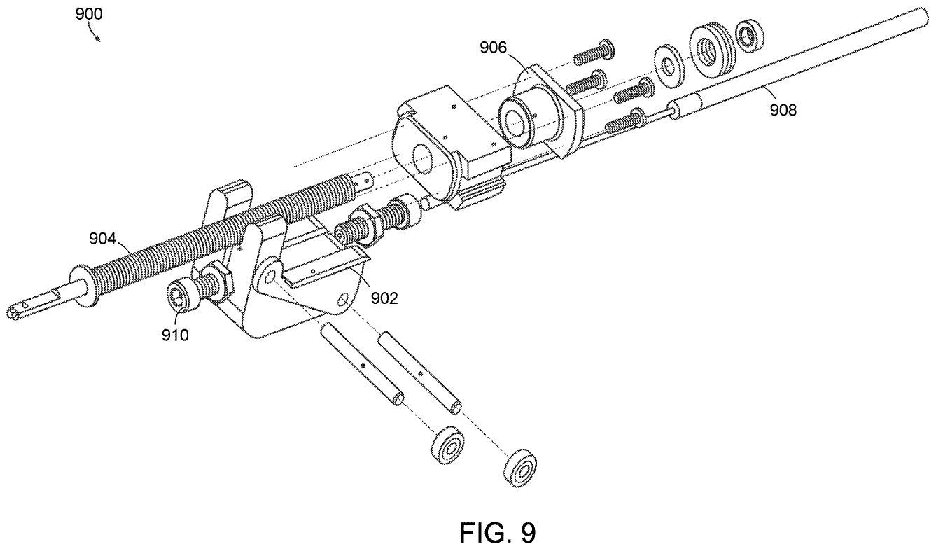

FIG. 9 illustrates an extension limiter 900 that can be used in an elbow orthosis to allow for elbow flexion without any input from the motor of the elbow orthosis. For example, the extension limiter 900 allows a user to flex his/her elbow while maintaining a set orthosis elbow angle. A sled 902 slides along a ball screw 904 that is part of a drivetrain (e.g., similar to the ball screw 808 of FIG. 8). Movement of the sled 902 along the ball screw 904 is prevented in one direction because a ball screw nut 906 blocks the sled 902 from moving. The sled 902 is free to move along the ball screw 904 away from the ball screw nut 906. User input can determine the position of the ball screw nut 906, limiting extension. The extension limiter 900 can also act as elbow support in conjunction with a brake mechanism (e.g., similar to the brake mechanism of FIG. 6) so that the elbow orthosis maintains a desired angle. A cable 908 (e.g., a Bowden cable) is connected to the sled 902 via a cable seat 910. For example, the cable 908 can be used to move an elbow orthosis, e.g., similar to the cable 710 of FIG. 7. The extension limiter 900 can act as a limit switch that automatically brakes the motor, e.g., as discussed further below. In some implementations, the extension limiter 900 can include a break-away mechanism that allows user's movement to exceed motor speed.

As discussed above, an elbow orthosis can include one or more surface EMG sensors that can detect EMG signals from a user. The detected EMG signals can control rotation of the elbow joint of the elbow orthosis. For example, a controller can receive the detected EMG signals and control a drive assembly of the elbow orthosis to bend the elbow joint.

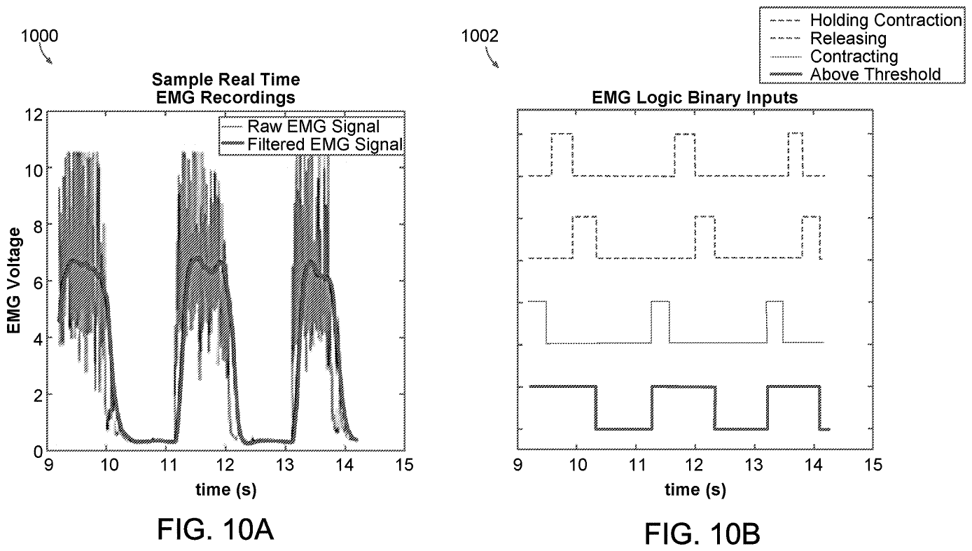

FIGS. 10A-B illustrate received EMG data and resulting signals of a controller. The plot 1000 illustrates raw EMG signals that were recorded using a differential amplifier. The raw EMG signal was amplified and bandpass filtered (30-300 Hz) and converted to a digital signal (e.g., using an NI USB 6361 device) and input into software for processing. The EMG signal was further low pass filtered (4 Hz) to create a linear envelope of the EMG (e.g., using LabVIEW's DAQmx package). The resulting waveform (illustrated in the plot 1002) was used as input to determine at which direction and velocity the motor of the elbow orthosis should turn. For example, two digital outputs can be sent from the controller to the motor to turn the motor on and off as well as to move the motor forward or backward.

To calibrate the controller, two calibration tests can be used. A quiescence test gathers a few seconds of baseline EMG. A threshold value for flexing the elbow can be determined by, e.g., the average baseline EMG value plus three standard deviations. A maximum voluntary contraction test can include a user flexing his/her arm to maximum exertion. The maximum EMG value can be used to proportionally control the speed of the motor of the elbow orthosis, e.g., controlling the speed at which the elbow orthosis bends. For example, if the EMG amplitude is at its maximum value (e.g., determined by the maximum voluntary contraction test) the output will have a duty cycle of 100%.

After calibration, the controller can receive the EMG signal envelope, limit switch positions, and angular position of the motor simultaneously. The EMG is approximately differentiated to determine whether the signal is rising, falling, staying the same, etc. If the derivative of the EMG signal is positive and the amplitude is above a threshold (e.g., determined by the quiescence test) then the controller moves the motor forward at a speed proportional to the EMG maximum value (e.g., determined by the maximum voluntary contraction test) to contract the elbow. If the derivative is negative, the motor is shut off, e.g., to maintain the position of the elbow orthosis or to straighten the elbow. If the derivative is close to zero and the amplitude is above the threshold, the brake is applied and the motor will hold the drivetrain at the current position, allowing the user to hold the current position of the orthosis without exertion. If the controller receives two quick pulses above threshold, the motor will reverse direction. If the motor activates one of the limit switches, the motor will stop. When this occurs, the user can unlock the brake (e.g., using a button to provide input) and the direction of the motor will reverse. The angular position of the motor can be used to determine proximity to the limit switches. If the motor is within 10% of the linear drive limits, the duty cycle of the output is reduced to 25% (if the duty cycle is not already below 25%). This prevents high speed collisions on either end of the worm drive.

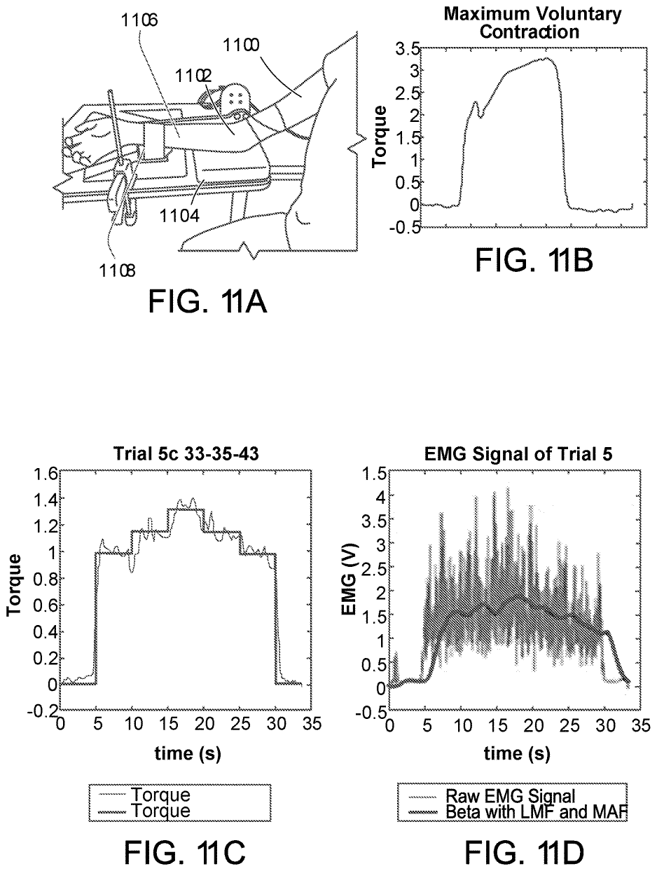

FIGS. 11A-D are plots of varying force levels and received EMG data from a patient with a brachial plexus injury (BPI). A fixture including a platform with a torque cell (e.g. a TS11—20 Nm Torque cell sold by Interface Inc.) and test support was built to collect patient muscle control data. FIG. 11A illustrates the patient 1100 positioned in the fixture with his/her elbow 1102 resting on the platform 1104 and his/her forearm 1106 secured to the fixture's arm 1108. The torque cell measures patient exertion, while a surface electrode (e.g., an MA-100 electrode sold by Motion Lab Systems, Inc.) placed on the biceps records muscle activity. Software (e.g., similar to the software described above) simultaneously acquires and records torque from the torque cell and EMG signals from the surface electrode. In the preliminary test illustrated in FIG. 11B, the patient was able to generate a maximum voluntary contraction. The maximum voluntary contraction was used to create a varying force protocol, illustrated in FIG. 11C. The patient was directed to generate varying force levels to follow the varying force protocol while the EMG output was collected (FIG. 11D). The illustrated data demonstrates feasibility of the proposed device and illustrates that a patient can modulate an EMG signal to control an elbow orthosis.

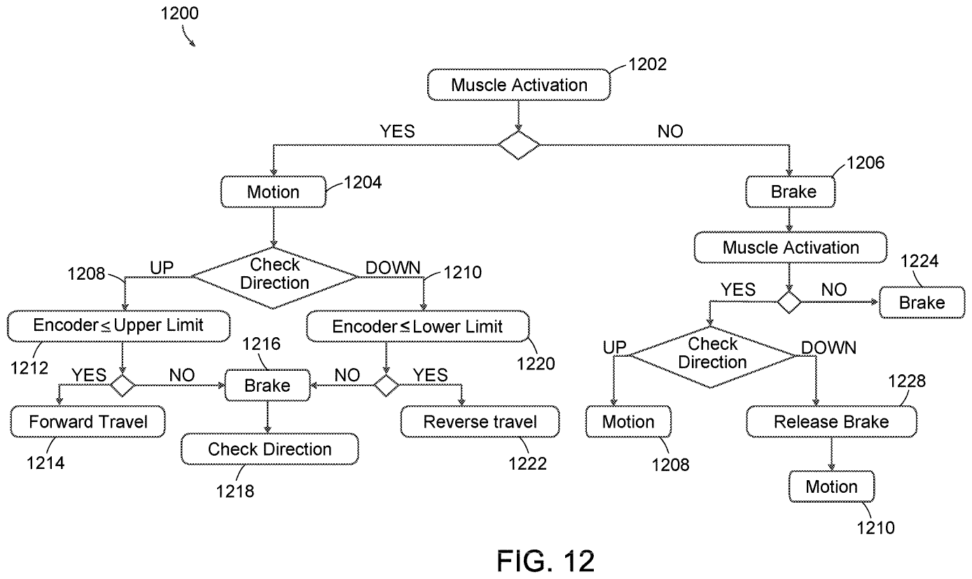

FIG. 12 illustrates a method 1200 of operating an elbow orthosis based on received EMG signals. For example, a controller of an elbow orthosis can perform the method 1200 to control the elbow orthosis 1200. The method 1200 includes determining whether muscle activation signals (e.g., EMG signals) are received 1202. If muscle activation signals are received, the controller can determine that the orthosis should move 1204. If muscle activation signals are not received, the controller can output a signal to activate a brake (e.g., similar to the brake 600 of FIG. 6) of the elbow orthosis 1206.

If the controller determines that the orthosis should move, the controller can determine in which direction the motor should move. For example, the motor can move in a forward direction or a reverse direction. The controller can determine that the motor should move in the forward direction 1208 (e.g., based on EMG signals as discussed above). Alternatively, the controller can determine that the motor should move in the reverse direction 1210 (e.g., based on EMG signals as discussed above).

If the controller determines that the motor should move in the forward direction, the controller can use the angular position of the motor (e.g., received from an encoder) to determine whether the motor will reach a limit switch 1212. If the motor is not near the limit switch, the controller will output a signal for the motor to move in the forward direction 1214. If the motor is near the limit switch, the controller will output a signal to activate the brake 1216 and then output a signal for the motor to change direction 1218, i.e., move in the reverse direction.

If the controller determines that the motor should move in the reverse direction, the controller can use the angular position of the motor to determine whether the motor will reach the opposite limit switch 1220. If the motor is not near the limit switch, the controller will output a signal for the motor to move in the reverse direction 1222. If the motor is near the limit switch, the controller will output a signal to activate the brake and then output a signal for the motor to change direction, i.e., move in the forward direction.

If the controller determines that no muscle activation signals are received at 1202, the controller the elbow orthosis should not move, the controller can output a signal to activate the brake 1206. After the brake is activated, the controller can continue to determine whether muscle activation signals are being received. As long as no activation signals are received, the brake remains activated 1224.

If muscle activation signals are received after the brake is activated, the controller can determine in which direction the orthosis should move. If the controller determines that the motor should move in the forward direction, the controller can use the angular position of the motor to determine whether the motor will reach a limit switch 1212, as discussed above. If the motor is not near the limit switch, the controller will output a signal for the motor to move in the forward direction 1214. If the motor is near the limit switch, the controller will output a signal to activate the brake 1216 and then output a signal for the motor to change direction 1218, i.e., move in the reverse direction.

If the controller determines that the motor should move in the reverse direction, the controller can output a signal to release the brake 1228. Once the brake is released, the controller can use the angular position of the motor to determine whether the motor will reach the opposite limit switch 1220. If the motor is not near the limit switch, the controller will output a signal for the motor to move in the reverse direction 1222. If the motor is near the limit switch, the controller will output a signal to activate the brake and then output a signal for the motor to change direction, i.e., move in the forward direction.

Testing of the elbow orthoses described above has been performed to assess elbow flexion improvements during activities of daily living. In one example, 11 subjects with brachial plexus injury were studied. A certified orthotist fit each subject with custom attachments for the orthosis. A surface EMG sensor was placed on the elbow flexor and signal activation thresholds were established, as discussed above. Two subjects were excluded from the study due to inadequate signal activation thresholds or technical issues. The other subjects completed regular activities for daily living with and without the orthoses. Motion capture technology was used to measure the subjects' kinematics. Joint coordinate system standards developed by the International Society of Biomechanics were used to obtain objective measurements of ranges of motion (e.g., elbow, shoulder, and trunk ranges of motion). The weight lifted and time for sustained elbow flexion were used to evaluate the contribution of the orthoses to upper extremity function.

All subjects were able to successfully flex and disengage the brake in the orthosis.

The orthosis improved elbow flexion range of motion by 23±18 degrees (p<0.001) and reduced compensatory movements at the shoulder (i.e., the humerothoracic joint) by 26±21 degrees (p<0.001) and the trunk by 7±14 degrees (p<0.05). The subjects were also able to perform tasks which they were unable to do without the orthoses. Six of the nine subjects carried a basket with a weight of 1.3±0.6 kg, while being unable to do so without wearing the orthoses. Additionally, the subjects were able to keep their elbows flexed for 1.3±3.17 seconds (p<0.01) longer while wearing the orthoses. These real-world results illustrate that the orthoses described above provide individuals with neuromuscular injuries better function during daily living activities and an improved quality of life.

A number of embodiments have been described. Nevertheless, it will be understood that various modifications may be made without departing from the spirit and scope of the disclosure. Accordingly, other embodiments are within the scope of the following claims.

1. An elbow joint of a powered orthosis comprising:
a battery;
an upper arm attachment configured to be worn on an upper arm of a user;
a forearm attachment configured to be worn on a forearm of the user;
a drivetrain configured to rotate the forearm attachment relative to the upper arm attachment, the drivetrain comprising:
a motor;
a belt drive;
a brake mechanism connected to the upper arm attachment and the forearm attachment; and
a torque mechanism comprising:
a pulley fixed to the forearm attachment;
a cable routed through the pulley, wherein the cable is fixed to the forearm attachment and the motor such that the cable provides constant torque to the forearm attachment;
an extension limiter configured to allow for elbow flexion, the extension limiter comprising:
a screw;
a nut positioned on the screw, wherein the position of the nut on the screw is variable based on user input; and
a sled attached to the cable and configured to move along the screw, wherein the sled is free to move in a first direction along the screw and is prevented from moving in a second direction along the screw by the nut;
a surface electromyography sensor configured to receive muscle signals of the user; and
a controller configured to receive input from the user and to control rotation of the forearm attachment relative to the upper arm attachment based on the muscle signals received by the surface electromyography sensor.

2. The elbow joint of claim 1, wherein the screw is a ball screw and the nut is a ball screw nut.

3. The elbow joint of claim 1, wherein the cable is a bowden cable.

4. The elbow joint of claim 1, wherein the battery, the motor, and the belt drive are contained within a housing.

5. The elbow joint of claim 4, wherein the housing is configured to be worn on a back of the user.

6. The elbow joint of claim 1, further comprising buttons, wherein the buttons are configured to receive input from the user to control the elbow joint.

7. The elbow joint of claim 6, wherein a first button allows the user to disengage the solenoid pin from the rotating cam.

8. The elbow joint of claim 7, wherein a second button allows the user to change the position of the nut.

9. The elbow joint of claim 1, wherein the sled allows for freedom of flexion movement.

10. The elbow joint of claim 1, wherein the sled allows user movement to exceed motor speed.

11. The elbow joint of claim 1, wherein the motor is located remotely and connected to the forearm attachment by the cable.

12. The elbow joint of claim 1, wherein the elbow joint is configured to generate a constant moment around the elbow joint.

13. The elbow joint of claim 1, wherein the elbow joint is configured to be worn on a left arm or a right arm.

14. The elbow joint of claim 1, wherein the forearm attachment is configured to extend due to gravity.

15. The elbow joint of claim 1, wherein the forearm attachment is configured to maintain a position when the motor is powered off.

16. The elbow joint of claim 1, further comprising additional extension limiters to protect from hyperextension.

17. The elbow joint of claim 1, wherein the brake mechanism comprises:
a rotating cam rotatably connecting the forearm attachment to the upper arm attachment; and
a solenoid pin configured to engage the rotating cam to prevent rotation of the rotating cam in one direction, wherein the rotating cam is free to rotate in the one direction when the solenoid pin disengages the rotating cam.

18. An elbow joint of a powered orthosis comprising:
a battery;
an upper arm attachment configured to be worn on an upper arm of a user;
a forearm attachment configured to be worn on a forearm of the user;
a drivetrain configured to rotate the forearm attachment relative to the upper arm attachment, the drivetrain comprising:
a motor, wherein the forearm attachment is configured to maintain a position when the motor is powered off,
a belt drive; and
a brake mechanism connected to the upper arm attachment and the forearm attachment, wherein the brake mechanism comprises:
a rotating cam rotatably connecting the forearm attachment to the upper arm attachment; and
a solenoid pin configured to engage the rotating cam to prevent rotation of the rotating cam in one direction, wherein the rotating cam is free to rotate in the one direction when the solenoid pin disengages the rotating cam.

19. The elbow joint of claim 18, wherein the battery, the motor, and the belt drive are contained within a housing.

20. The elbow joint of claim 19, wherein the housing is configured to be worn on a back of the user.
